(19) United States
(12) Patent Application Publication
(10) Pub. No.: US 2026/0216099 A1
(43) Pub. Date: Jul. 30, 2026

(54) BROMOQUINONE AS A TREATMENT FOR OBESITY AND CANCER

(71) Applicants: Brandon Yu, Greenwich, CT (US); Chang-An Yu, Stillwater, OK (US); Claire Yu, Greenwich, CT (US)

(72) Inventors: Brandon Yu, Greenwich, CT (US); Chang-An Yu, Stillwater, OK (US); Claire Yu, Greenwich, CT (US)

(21) Appl. No.: 19/147,381

(22) PCT Filed: Feb. 12, 2024
(86) PCT No.: PCT/US2024/015370

§ 371 Date: Jul. 11, 2025

Related U.S. Application Data
(60) Provisional application No. 63/484,559, filed on Feb. 13, 2023.

(51) Int. Cl. A61K 31/122; A61P 3/04; A61P 35/00; A61P 35/02; C07C 50/28

(52) U.S. Cl. CPC A61K 31/122; A61P 3/04; A61P 35/00; A61P 35/02; C07C 50/28

(57) ABSTRACT

The present invention is directed to compounds that can treat obesity or cancer, and more particularly to 6-bromo-decyl-benzoquinone and derivatives as treatments for obesity and various forms of cancer.

### CROSS-REFERENCE TO RELATED APPLICATIONS

This application claims the benefit of U.S. Provisional Application Ser. No. 63/484,559 filed Feb. 13, 2023, which is incorporated by reference herein in its entirety.

### STATEMENT OF GOVERNMENT SUPPORT

This invention was made in part with government support from the National Institutes of Health Grant No. GM30721. The United States government has certain rights in this invention.

### BACKGROUND OF THE INVENTION

### Field of the Invention

The present invention is directed to compounds that can treat obesity or cancer, and more particularly to 6-Br-ubiquinone and derivatives as treatments for obesity and various forms of cancer.

### Brief Description of the Related Art

More than 90% of the energy required to sustain life, growth, and the structural integrity of a living organism is derived from a process called oxidative phosphorylation (1,2). This process takes place in the mitochondrial inner membrane of eukaryotic cells or in the cytoplasmic membrane of prokaryotic organisms, via a coupling reaction of two multi-subunit membrane protein complexes: the electron transfer chain complexes (Complexes I (3,4), II (5,6), III (7,8) and IV (9,10)), and the ATP synthase complex (Complex V) (11). The electron transfer chain complexes catalyze the oxidation of NADH and succinate generated from the Krebs cycle. Among the four electron transfer complexes, Complexes I, III, and IV generate a proton gradient but Complex II does not. FIG. 1 illustrates electron transport Complexes I to IV and ATP synthase (Complex V) in the mitochondrial inner membrane.

Coenzyme Q₁₀ (2,3-dimethoxy-5-(isoprenyl)₁₀-6-methyl-1,4-benzoquinone) (12) (FIG. 2A) mediates electron transfer between complexes I or II and III and is the only redox component in the electron transfer system whose concentration can be manipulated by food intake. The study of coenzyme Q₁₀ is extremely challenging, however, due to its low solubility. Fortunately, researchers in the 1970s (12,13) synthesized various coenzyme Q₁₀ derivatives with shorter alkyl side chains to increase their solubility and yet still maintain their electron transfer activity. The electron transfer activity of these synthetic derivatives depends on the carbon chain length of the alkyl group of coenzyme Q, with a length of 10 carbons having the highest transfer ability (much shorter than 50 found in the natural coenzyme Q₁₀). The most popular synthetic derivative is known as decyl-benzoquinone (DBQ, or Q₀C₁₀) (12,14) which has a ten-carbon alkyl side-chain. Q₀C₁₀ was used extensively in mitochondrial bioenergetic research. FIG. 2B shows the chemical structure of Q₀C₁₀. The spectral and redox properties of Q₀C₁₀ are identical to those of native coenzyme Q₁₀ with an absorption maximum at 277 nm and a midpoint redox potential of 110 mV. Additional compounds that affect the electron transfer system could be advantageous in treatment of obesity and related diseases such as cancers.

### SUMMARY OF THE INVENTION

Since obesity (15,16) is a medical condition that results from excess body fat accumulation due to the oversupply of energy, reduction of the energy supply or recovery efficiency could be an effective way to reduce obesity. Although preliminary results on isolated mitochondria indicate that bromoquinone can reduce energy coupling efficiency, the effect of this compound on growth rate or body weight increases in the tissue cultures (cell lines) and organisms has not yet been investigated.

The present invention is based on the discovery that a modified variant of coenzyme Q called bromoquinone (6-Br-Q₀C₁₀) has been found to have significantly less electron transfer activity than native coenzyme Q. It reduces the energy coupling efficiency by 30%, which suggests that it could be useful in treating obesity. The effect of bromoquinone on several cell lines was studied using three main detection methods and found that bromoquinone reduces the growth of all cell lines tested. Moreover, the effects are particularly apparent in diabetic cells and cancer cells. These results support the idea of using bromoquinone as a treatment to treat obesity or to slow the spread of cancerous tumors.

Replacing the 6-methyl group of the benzoquinone ring of Q₀C₁₀ with a bromine atom creates 6-Bromo-decyl-1,4-benzoquinone (6-Br-Q₀C₁₀) which is referred to as bromoquinone. FIG. 2C and Formula I show the chemical structure of bromoquinone (6-Br-Q₀C₁₀, Formula I).

Introducing an electron-withdrawing group such as bromine should have an effect on the redox potential and UV spectrum, and this is indeed the case. Bromoquinone has a higher redox potential than that of Coenzyme Q₁₀, by about 30 mV and UV spectral shift by 24 nm. A decrease of approximately 30% of energy conservation efficiency has been observed in isolated mitochondria that were treated with bromoquinone. This decrease may be due to the loss of energy conservation site II at Complex III in the bromoquinone-treated mitochondria (2).

Accordingly, in one aspect, the present invention is directed to a compound of Formula I

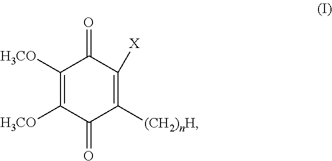

or a salt, solvate, or prodrug thereof, wherein n is an integer from 1 to 15, and X is a halogen.

In another aspect, the present invention is directed to a pharmaceutical composition comprising the compound of Formula I or a salt, solvate, or prodrug thereof, together with a pharmaceutically acceptable carrier.

In another aspect, the present invention is directed to a method of treating a disease, disorder, or medical condition in a patient, comprising the step of providing to a patient in need thereof a pharmaceutical composition comprising the compound of Formula I or a salt, solvate, or prodrug thereof, together with a pharmaceutically acceptable carrier.

These and other aspects will be described in the following written description and accompanying drawings.

### BRIEF DESCRIPTION OF THE DRAWINGS

FIG. 1 illustrates electron transport complexes I-IV and ATP synthase (complex V) in the mitochondrial inner membrane (adopted from Chimia International Journal for Chemistry 72, 291-296, 2018).

FIGS. 2A, 2B, and 2C illustrate chemical structures of the disclosed embodiments. FIG. 2A illustrates the chemical structure of coenzyme Q₁₀ (2,3-dimethoxy-5-(isoprenyl)10-6-methyl-1,4-benzoquinone); FIG. 2B illustrates the chemical structure of Q₀C₁₀ (2,3-dimethoxy-5-decyl-6-methyl-1,4-benzoquinone); and FIG. 2C illustrates the chemical structure of bromoquinone (2,3-dimethoxy-5-decyl-6-Br-1,4 benzoquinone).

FIGS. 3A and 3B illustrate the effect of bromoquinone on cell growth of L-929, Raw 264-7 and THP-1 cells. FIG. 3A illustrates the inhibition of cell growth with increased concentrations of bromoquinone, and FIG. 3B shows the inhibition of cell attachment with increased concentrations of bromoquinone.

FIGS. 4A, 4B, and 4C illustrate the effect of bromoquinone on cell growth of HEK293, HeLa, INS-1, and ADSC cells. FIG. 4A shows the inhibition of cell growth with increased concentrations of bromoquinone as measured by counting viable and nonviable cells using Trypan Blue and a hemocytometer. FIG. 4B shows cell viability as determined by MTT cytotoxicity assay. FIG. 4C shows images of HEK293 cells after 48-hour treatment with increased concentrations of bromoquinone. Cells were stained with bisbenzimide.

FIGS. 5A and 5B show the growth (weight gain) from Nov. 5, 2023 to Feb. 3, 2024 of the three groups of rats, individually (FIG. 5A) and as group average (FIG. 5B).

FIG. 6 illustrates weight gain of control, high-fat carrier, and 6-Br-Q₀C₁₀ treated rats since Jan. 16, 2024. Open bar (left) is the control group, slashed bar (center) is the high-fat carrier only group, and the hatched bar (right) is the 6-Br-Q₀C₁₀ in high-fat carrier group.

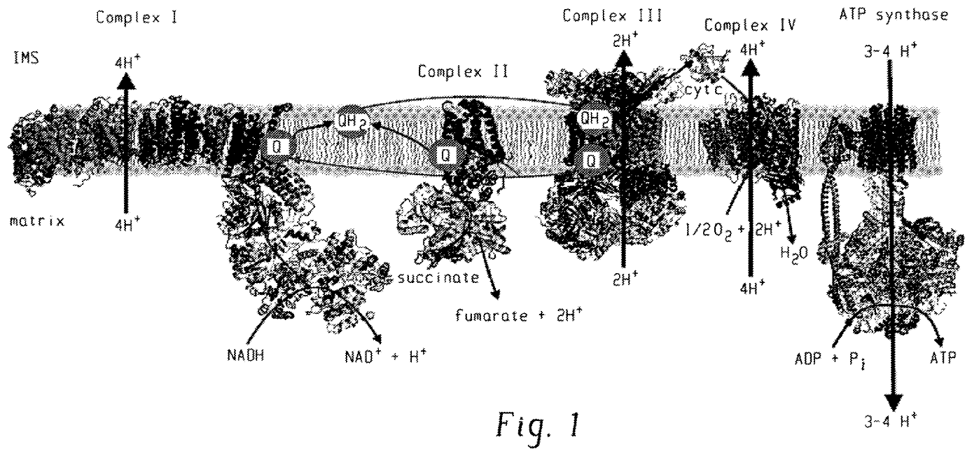

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸P, ¹⁵N, ¹⁸O, ⁷⁶Br, ¹²⁵I and ¹³¹I.

All formulae disclosed herein include all salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“−”) that is not between two letters or symbols is used to indicate a point of attachment for a substituent.

“Alkyl” includes both branched and straight chain saturated aliphatic hydrocarbon groups, having the specified number of carbon atoms, generally from 1 to about 15 carbon atoms. The terms C₁₋₆ alkyl, C₁-C₆ alkyl and C₁-C₆ alkyl as used herein all indicate an alkyl group having from 1, 2, 3, 4, 5, 6 or up to 15 carbon atoms. Other embodiments include alkyl groups having from 1 to 8 carbon atoms, 1 to 4 carbon atoms or 1 or 2 carbon atoms, e.g. C₁₋₈ alkyl, C₁-4 alkyl, and C₁₋₂ alkyl. When Con alkyl is used herein in conjunction with another group, for example, —C₀₋₄ alkyl(phenyl), the indicated group, in this case phenyl, is either directly bound by a single covalent bond (C₀ alkyl), or attached by an alkyl chain having the specified number of carbon atoms, in this case 1, 2, 3, or 4 carbon atoms. Alkyls can also be attached via other groups such as heteroatoms as in —OC₀₋₄ alkyl(C₃₋₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula I, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers (e.g., asymmetric carbon atoms), stereogenic axes, rotamers with restricted rotation (e.g., atropisomers) and the like, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, prodrugs, free base compound and salts) of the compounds of the invention may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

The term “solvate” refers to a chemical complex formed by the interaction of a solvent and a solute, such as the chemical compounds of the present invention.

The term “prodrug” refers to a biologically inactive compound which can be metabolized inside the body to produce a drug.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, B. and Wilen, S., Stereochemistry of Organic Compounds (1994) John Wiley & Sons, Inc., New York, Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Salts” include derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts. In an embodiment, the compounds of the present invention are synthesized or isolated as trifluoroacetic acid (TFA) salts.

In one embodiment, the salt forms of the compounds of the present invention described above may include pharmaceutically acceptable salts. Examples of pharmaceutically acceptable salts include, but are not limited to, non-toxic mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

As indicated above, in one aspect, the present invention is directed to a compound of Formula I

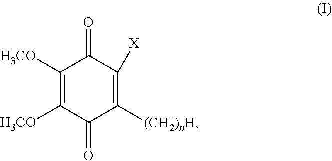

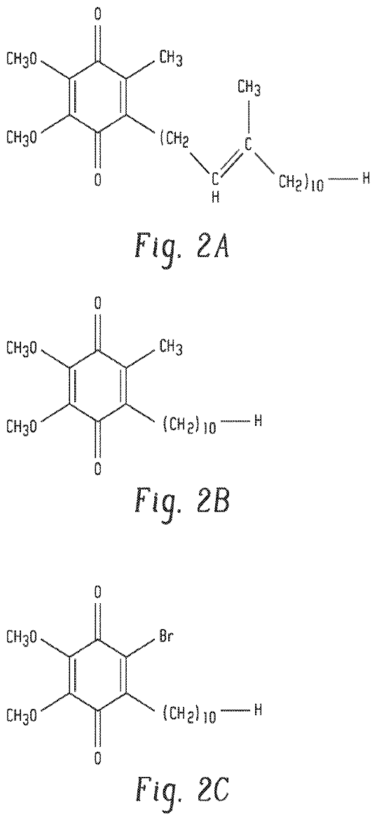

or a salt, solvate, or prodrug thereof, wherein n is an integer from 1 to 15, and X is a halogen. FIG. 2C and Formula I show the chemical structure of 6-bromo-decyl-1,4-benzoquinone (termed bromoquinone and abbreviated herein as 6-Br-Q₀C₁₀). Applicants have discovered that introducing an electron-withdrawing group such as bromine to Q₀C₁₀ has an effect on the redox potential and UV spectrum, thus making 6-Br-Q₀C₁₀ a useful treatment for obesity and cancers.

Compounds disclosed herein can be administered to a patient as the neat or freebase chemical, but are preferably administered as a pharmaceutical composition. Accordingly, the invention encompasses pharmaceutical compositions comprising a compound or a salt (including a pharmaceutically acceptable salt) of a compound, such as a compound of Formula I, together with at least one pharmaceutically acceptable carrier. The pharmaceutical composition may contain a compound or salt of Formula I as the only active agent, but is preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example, the pharmaceutical composition may contain a molar ratio of about 0.5:1, about 1:1, about 2:1, about 3:1 or from about 1.5:1 to about 4:1 of an additional active agent to a compound of Formula I.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt %) of a compound of Formula I and usually at least about 5 wt % of a compound of Formula I. Some embodiments contain from about 25 wt % to about 50 wt % or from about 5 wt % to about 75 wt % of the compound of Formula I.

### Treatment Methods

The compounds of Formula I, as well as pharmaceutical compositions comprising the compounds, are useful for diagnosis or treatment of a disease, disorder, or medical condition relating to obesity and various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

According to the present invention, a method of treating obesity or cancer diseases or conditions comprises providing to a patient in need of such treatment a therapeutically effective amount of a compound of Formula 1. In one embodiment, the patient is a mammal, and more specifically a human. As will be understood by one skilled in the art, the invention also encompasses methods of treating non-human patients such as companion animals, e.g. cats, dogs, and livestock animals.

A therapeutically effective amount of a pharmaceutical composition is preferably an amount sufficient to reduce or ameliorate the symptoms of a disease or condition. In the case of obesity or cancer-mediated diseases for example, a therapeutically effective amount may be an amount sufficient to reduce or ameliorate obesity or cancer. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound of Formula I to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered singularly (i.e., sole therapeutic agent of a regime) to treat or prevent obesity or cancer-mediated diseases and conditions, or may be administered in combination with another active agent. One or more compounds of Formula I may be administered in coordination with a regime of one or more other active agents such as anticancer cytotoxic agents. In an embodiment, a method of treating or diagnosing cancer in a mammal includes administering to said mammal a therapeutically effective amount of a compound of Formula I, optionally in combination with one or more additional active ingredients.

As will be appreciated by one skilled in the art, the methods of treatment provided herein are also useful for treatment of mammals other than humans, including for veterinary applications such as to treat horses and livestock, e.g. cattle, sheep, cows, goats, swine and the like, and pets (companion animals) such as dogs and cats.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g. blood, plasma, serum, cellular interstitial fluid, saliva, feces, and urine) and cell and tissue samples of the above subjects will be suitable for use.

In one embodiment, the invention provides a method of treating a disease, disorder, or medical condition including obesity and various cancers, in a patient identified as in need of such treatment, the method comprising providing to the patient an effective amount of a compound of Formula I. The compounds of Formula I provided herein may be administered alone, or in combination with one or more other active agents.

In another embodiment, the method of treating obesity or cancers may additionally comprise administering the compound of Formula I in combination with one or more additional compounds, wherein at least one of the additional compounds is an active agent, to a patient in need of such treatment. The one or more additional compounds may include additional therapeutic compounds, including anticancer therapeutic compounds such as doxorubicin, paclitaxel, docetaxel, cisplatin, camptothecin, temozolomide, avastin, Herceptin, Erbitux, and the like.

### EXAMPLES

### Materials

Synthesis of 6-bromo-2, 3-dimethoxy-5-alkyl-1, 4-benzoquinones and 6-chloro-2, 3-dimethoxy-5-alkyl-1, 4-benzoquinones. Scheme 1 illustrates the method used in the synthesis of those 6-bromo- and 6-chloro-Q derivatives.

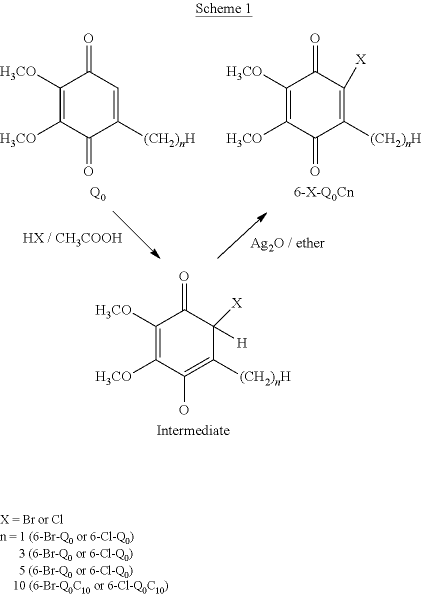

6-Bromo-2,3-dimethoxy-5-methyl-1, 4-benzoquinone (6-Br-Q₀) was synthesized as follows: One hundred and eighty (180) mg of Q₀ (2,3-dimethoxy-5-methyl-1,4-benzoquinone, 1 mmol) in 5 ml of acetic acid was mixed with 5 ml of 48% hydrobromic acid. The mixture was stirred at room temperature for 30 minutes, then diluted with 40 ml of water and extracted with ether. The ether extract was washed with H₂O and dried over anhydrous Na₂SO₄. One gram of Ag₂O was added to the dried ether solution and the mixture was stirred for one hour at room temperature. The solid phase was removed by filtration, and the filtrate solution was dried in a rotary evaporator. The crude product was purified by TLC, yield 92%, 6-Br-Q₀ is a red crystalline, m. p. 58-60° C. 1H NMR (CDCl₃): 2.20 (s, 3H), 4.02 (s, 3H), 4.06 (s, 3H) ppm. UV^{EtOH}: oxid, 302 nm; red, 293 nm. MS (m/z), 260 (M), 262 (M+2); HRMS, 259.9681 (cal. 259.9684).

By using the same method, a series of 6-bromo- and 6-chloro-Q derivatives were synthesized. Starting materials, such as 2,3-dimethoxy-1,4-benzoquinone, 2,3-dimethoxy-5-propy-1,4-benzoquinone, 2,3-dimethoxy-5-pentyl-1,4-benzoquinone, and 2,3-dimethoxy-5-decyl-1,4-benzoquinone, were prepared according to the methods described previously (22, 23).

6-Bromo-2, 3-dimethoxy-5-propyl-1, 4-benzoquinone (6-Br-Q₀C₃): Red oil. 1H NMR (CDCl₃): 1.02 (t, 3H), 1.75 (m, 2H), 2.20 (t, 2H), 4.02 (s, 3H), 4.06 (s, 3H) ppm. UV^{EtOH}: oxid, 302 nm; red, 293 nm. MS (m/z): 288 (M); 290 (M+2).

6-Bromo-2, 3-dimethoxy-5-pentyl-1, 4-benzoquinone (6-Br-Q₀C₅): Red oil. ¹H NMR (CDCl₃): 0.89 (t, 3H), 1.28-1.75 (m, 6H), 2.20 (t, 2H), 4.02 (s, 3H), 4.06 (s, 3H) ppm. UV^{EtOH}; oxid, 302 nm; red, 293 nm. MS (m/z): 316 (M); 318 (M+2).

6-Bromo-2, 3-dimethoxy-5-decyl-1, 4-benzoquinone (6-Br-Q₀C₁₀): Red oil. JH NMR (CDCl₃): 0.89 (t, 3H), 1.23-1.6 (m, 16H) 2.20 (L, 2H), 4.02 (s, 3H), 4.06 (s, 3H) ppm. UV^{EtOH}: oxid, 302 nm; red, 293 nm. MS (m/z): 386 (M); 388 (M+2); HRMS, 386.1098 (cal. 386.1093).

6-Chloro-2, 3-dimethoxy-5-methyl-1,4-benzoquinone (6-Cl-Q₀): Red crystalline. 1H NMR (CDC) 3): 2.18 (s, 3H), 4.01 (s, 3H), 4.05 (s, 3H) ppm. UV^{EtOH}: oxid, 287 nm; red, 290 nm. MS (m/z), 216 (M), 218 (M+2); HRMS, 216.0185 (cal. 216.0190).

6-Chloro-2, 3-dimethoxy-5-propyl-1,4-benzoquinone (6-Cl-Q₀C₃): Red oil. ¹H NMR (CDCl₃): 0.95 (t, 3H), 1.62 (m, 2H) 2.16 (t, 2H), 4.01 (s, 3H), 4.04 (s, 3H) ppm. UV^{EtOH}: oxid, 287 nm; red, 290 nm. MS (m/z): 244 (M); 246 (M+2).

6-Chloro-2, 3-dimethoxy-5-pentyl-1,4-benzoquinone (6-Cl-Q₀C₅): Red oil. ¹H NMR (CDCl₃): 0.89 (t, 3H), 1.20-1.62 (m, 6H), 2.15 (t, 2H), 4.01 (s, 3H), 4.04 (s, 3H) ppm. UV^{EtOH}: oxid, 287 nm; red, 290 nm. MS (m/z): 272 (M); 274 (M+2).

6-Chloro-2, 3-dimethoxy-5-decyl-1, 4-benzoquinone (6-Cl-Q₀C₁₀): Red oil. ¹H NMR (CDCl₃): 0.89 (t, 3H), 1.18-1.60 (m, 16H), 2.14 (t, 2H), 4.01 (s, 3H), 4.04 (s, 3H) ppm. UV^{EtOH}: oxid, 7 nm; red, 290 nm. MS (m/z): 342 (M); 344 (M+2).

Crude bromoquinone was further purified to homogeneity using thin-layer chromatography. The purity of bromoquinone was confirmed by its absorption peak at 303 nm and the concentration was determined spectrophotometrically, using a millimolar extinction coefficient of 20 (14).

Synthesis of bromoquinone (6-Br-Q₀C₁₀) can also be performed using the following alternate method:

0.25 ml concentrated H₂SO₄ was dissolved in 25 ml of acetic acid, and 0.4 ml of 2,3-dimethoxyphenol (2.5 mmol) was added with stirring. 9.3 ml H₂O₂ (30%) was added dropwise with stirring over 15 min), and allowed to stir for 60 min. 30 ml water were added and extracted with CH₂Cl (40 ml×3) to yield the 6-H-Q₀ solution. CH₂Cl₂ was removed and crude 6-H-Q₀ was purified by TLC or column chromatography (Ether:Hexane=1:3) to get pure 6-H-Q₀.

Purified 6-H-Q₀ 0.3 mmol was dissolved in 9 ml CH₃CN and 4 ml H₂O. 0.56 g of CH₃(CH₂)₉COOH (3 mmol) was added and the mixture heated to 60-64° C. 0.4 g AgNO₃ and 0.8 g K₂S₂O₈ were added and stirred for 24 min at 60-64° C. 30 ml H₂O were added and extracted with ether. The ether layer was washed with water, 2% Na₂CO₃ and water, and the ether layer was removed. The resulting 6-H-Q₀C₁₀ compound was purified by TLC (Ether:Hexane=1:3) to get pure 6-H-Q₀C₁₀.

Purified 6-H-Q₀C₁₀ 0.4 mmol was dissolved in 5 ml of CH₃COOH, and an HBr solution 2.5 ml CH₃COOH and 4 ml HBr (48%) was added dropwise and stirred for 1 hour. 15 ml H₂O were added and extracted with ether. The ether layer was washed with H₂O and dried with anhydrous Na₂SO₄, 0.2 g Ag₂O was added to the dried ether layer, stirred for 3 hours, and the solid phase was removed by filtration. Ether was removed from the crude 6-Br-Q₀C₁₀ product and purified by TLC (Ether:Hexane=1:3) to yield pure 6-Br-Q₀C₁₀.

### Cell Line Studies

Commercially-available cell lines and their corresponding culture media were used in this investigation. DMEM and RPMI media were purchased from ThermoFisher Scientific or Sigma-Aldrich. Bisbenzimide was purchased from FisherScientific. Cell Counting Kit-8 (CCK-8) reagents were obtained from Boster Bio. The MTT assay kit (cb211091) was from Abcom. The cell lines are summarized in Table 1.

TABLE 1
Cell Lines, Their Origin and Culture Media

| Cell lines | Cell type | Organism | Morphology | Tissue | Medium Used |
|---|---|---|---|---|---|
| rMc-1 | Rat retinal muller | Rat | Epithelial | Eye; Choroid; | DMEM |
| | cell | | | Retina | |
| ARPE- | Human retinal | Human | Epithelial | Eye; Retina, Retinal | DMEM |
| 19 | pigment epithelial | | | pigmented | |
| | cell | | | epithelium | |
| HepG2 | Human hepatocyte | Human | Epithelial-like | Liver | DMEM |
| L929 | Mouse fibroblast | Mouse | Fibroblast | Subcutaneous | ATCC CCL |
| | cells | | | connective tissue; | |
| | | | | Adipose; Areolar | |
| Raw264- | Mouse | Mouse | Monocyte/ | Ascites | MEM& |
| 7 | macrophage | | macrophage | | |
| THP1 | Monocyte of | Human | Monocyte | Peripheral blood | RPM1 |
| | human leukemia | | | | |
| HEK293 | Human kidney cell | Human | Epithelial | Kidney; Embryo | DEME |
| HeLa | Human cervical | Human | Epithelial | Uterus; Cervix | DEME |
| | cancer cells | | | | |
| INS-1 | Rat insulinoma | Rat | Epithelial | Pancreas; Islet of | RPM1 |
| | cells | | | Langerhans | |
| ADSC | Adipose-derived | Human | Spindle-shaped, | Adipose tissue | MesenPRO |
| | mesenchymal stem | | fibroblast-like | | RS |
| | cells | | | | |

### Methods

Three methods were used to determine cell growth after bromoquinone treatment and incubation, and cell imaging was performed using bisbenzimide-based nuclear fluorescent staining.

Cell Counting Assay: For the cell counting assay, rMc-1, ARPE-19, and HepG2 cells were cultured in DMEM with 4.5 g/L glucose supplemented with 10% fetal bovine serum (FBS), penicillin, and streptomycin. These cells were seeded at 50% confluency before bromoquinone treatment. About 5,000 cells were seeded in a 24-well plate and treated with various amounts of bromoquinone and incubated at 37° C. under 5% CO₂. Cell growth was examined by cell counting stained with Trypan Blue. The Raw264.7 and L929 cells were cultured in DMEM supplemented with 10% (vol/vol) heat-inactivated FBS (SH30910.03; Hyclone) at 37° C. and 5% CO₂. THP-1 cells were cultured in RPMI-1640 medium supplemented with 10% (v/v) heat-inactivated FBS at 37° C. and 5% CO₂.

Cell Attachment Assay: For the cell attachment assay, Raw264.7 and L929 cells were pre-seeded (100,000 cells/well) in 12-well plates (overnight incubation) at 37° C. and 5% CO₂. Bromoquinone was added 24 hours after pre-seeding, and cell attachment was determined by counting attached cells at 72 hours post-treatment. THP-1 cells were pre-seeded (100,000 cells/well) in the presence of 320 nM phorbol-12-myristate-13-acetate (PMA) in 12-well plates (overnight incubation) at 37° C. and 5% CO₂. Bromoquinone was added 24 hours after pre-seeding, and cell attachment was determined by counting attached cells at 72 hours post-treatment. Raw264.7, L929, and THP-1 cells were cultured in 12-well plates (100,000 cells/well) in the presence of bromoquinone for 72 hours at 37° C. and 5% CO₂. The live cell number was determined by Trypan Blue staining. HEK293 and HeLa cells were cultured in DMEM supplemented with 10% (v/v) fetal bovine serum and 1% (v/v) penicillin-streptomycin. INS-1 cells were cultured in RPMI 1640 media supplemented with 10% (v/v) FBS and 1% (v/v) penicillin-streptomycin. All the cells were incubated at 37° C. and 5% CO₂ atmosphere in 10-cm plates. When the cells reached about-80% confluency, they were harvested and cultured on 96-well plates with 5×10³ cells seeded per well. For each cell line, three 96-well plates were prepared—one for MTT assay and two for bisbenzimide-based nuclear fluorescent stain assay. After 24-hour incubation, the culture medium was replaced by the same medium containing various concentrations (0, 4, 8, 16, 32, and 64 μM) of bromoquinone. Three replicates were made for each condition. The plates then continued to be incubated for another 24 hours (two plates) or 48 hours (one plate). One plate with 24-hour treatment was used for the MTT cell viability assay. The other 24-hour treatment plate and the 48-hour treatment plate were used for the Bisbenzimide-based nuclear fluorescent stain cell imaging.

MTT Cell Viability Assay: The MTT assays were performed using the MTT Assay kit. Ten μL of the kit reagent were added into each well, and optical absorption (OD) at 450 nm was measured using a multifunction microplate reader (Infinite M200 Pro, Tecan) after incubation for 2 hours at 37° C. The cell viability is expressed in the percentage of OD of treated cells versus that of untreated cells.

Bisbenzimide-based Nuclear Fluorescent Stain and Cell Imaging: After the 24- and 48-hour bromoquinone treatment, the cells in the 96-well plates were used for bisbenzimide-based nuclear fluorescent stain. Cell imaging was first fixed with 2% paraformaldehyde solution for 20 minutes at room temperature, and then bisbenzimide (0.5 mg/mL) was added to each well to a final concentration of 1.0 μg/mL. The cells were then incubated at room temperature for 15 minutes before the cell imaging analysis. Cell images were taken with a Zeiss AXIO Observer. Z1 Fluorescence Motorized Microscope w/Definite Focus.2 Pred 7. Zen 3.1 (Zen Pro) software was used.

### Results

When the cell lines (rM-1, ARPE-19 and Hep G2) were cultured for 48 hours in their corresponding media in the presence of various concentrations (0, 0.5, 2.5. 5, and 10 μM) of bromoquinone, the cell survival rate decreased as the concentration of bromoquinone increased. When the concentration of bromoquinone was 10 μM, the cell survival rate decreased by 70-80%, while under the same conditions, no effect on cell growth was observed if normal coenzyme Q or Q₀C₁₀ was used.

The effect of bromoquinone on Hep-G2 is very small, even at 10 μM bromoquinone. Without being bound by any particular theory, one possible reason is that the Hep-G2 cells might have a high concentration of native coenzyme Q, so a higher concentration of bromoquinone might be needed in order to compete against native coenzyme Q to result in higher inhibition. Table 2 below shows the results in tabular form.

TABLE 2
Effect of Bromoquinone on the Growth
of rMC-1, ARPE19 and Hep-G2 Cells

| | Bromoquinone Concentration | | | | | |
|---|---|---|---|---|---|---|
| | 0 μM | 0.5 μM | 1 μM | 2.5 μM | 5 μM | 10 μM |
| rMC-1 | 100% (1.2 × 10⁶) | 88.2 | 80.1 | 68.1 | 36.2 | 27.9 |
| ARPE19 | 100% (3.1 × 10⁵) | 85.4 | 80.9 | 60.0 | 41.9 | 26.6 |
| Hep G2 | 100% (1.3 × 10⁶) | 103.1 | 107.8 | 104.2 | 98.1 | 93.3 |

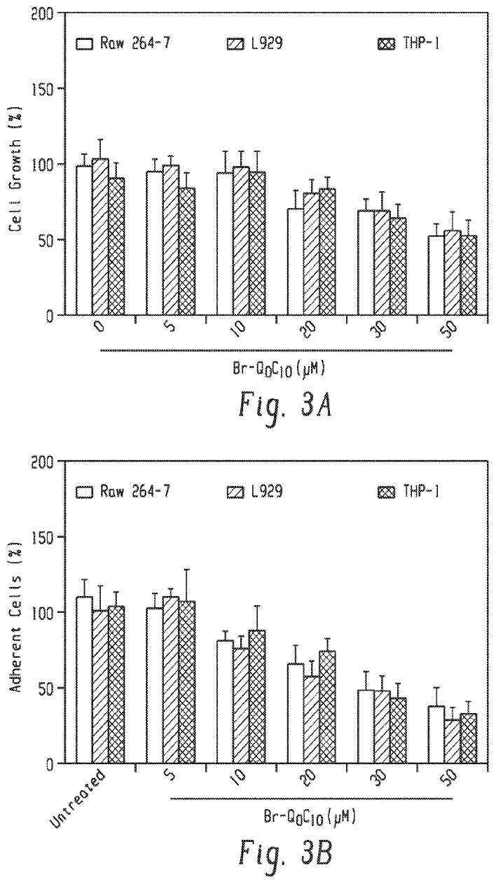

FIG. 3 below shows the effect of bromoquinone on cell growth of L-929, Raw 264-7 and THP-1 cells, which were cultured under the same conditions in the presence of bromoquinone. The concentrations of bromoquinone used were 0, 5, 10, 20, 30, and 50 μM. Bromoquinone inhibition of cell growth is concentration-dependent. Up to 50% inhibition of cell growth (FIG. 3A) was observed when the concentration reached 50 μM. Using the same bromoquinone concentration, a 70% inhibition (FIG. 3B) was observed for cell attachment.

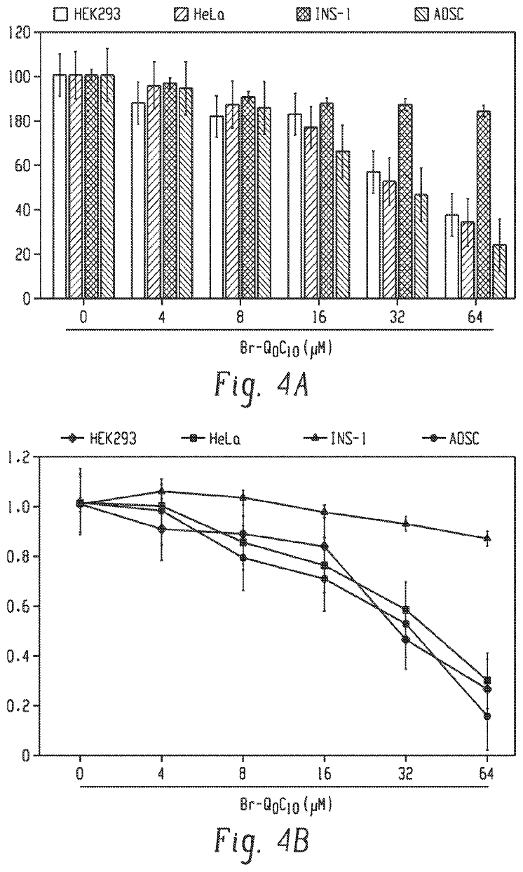

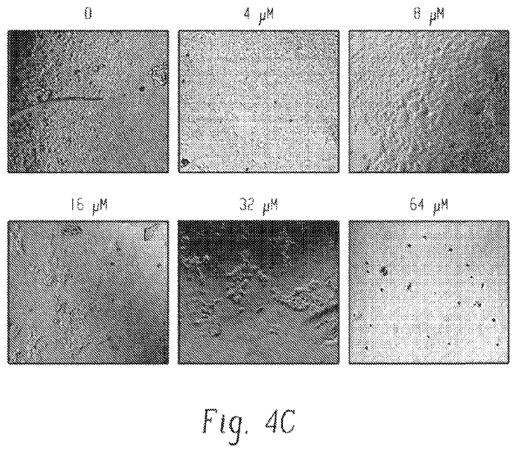

FIG. 4 shows the effects of bromoquinone on cell lines HEK-293, HeLa, INS-1, and ADSC, using three different detection methods: cell counting (FIG. 4A), MTT assay (FIG. 4B), and cell imaging (FIG. 4C). As described above, cell lines were cultured in their corresponding media in the presence of various concentrations of bromoquinone (0, 2, 4, 8, 16, 32, and 64 μM) for 48 and 72 hours. After 48 hours of treatment, inhibitions of 69%, 66%, and 82% were observed for HEK-293, HeLa, and ADSC, respectively (FIG. 4A). Similar results were obtained using the MTT assay (FIG. 4B). As expected, when the incubation was prolonged to 72 hours, more inhibition was observed. Inhibitions of 95%, 97%, and 90% were observed for HEK-293, HeLa, and ADSC, respectively, after 72 hours. The effect of bromoquinone on the INS-1 cell line, however, was much more muted, as less than 20% inhibition was observed at the highest concentration tested.

### Discussion

The obesity epidemic is one of the most serious health problems that our country faces today. Obesity is commonly defined as having a BMI (Body Mass Index) of over 30, and a BMI between 25-30 is classified as overweight (15, 16). According to Medical Laboratory Observer, obesity affects over 107 million Americans (a figure representing over 33% of the American population) and costs Americans over $147 billion annually (15,16). Medical treatment of obesity is difficult, and there are few good options. Surgery is risky, and there are only a few effective medications available. Although a significant number of specific diets aiming to reduce food intake are available, none are effective in curing obesity because they are all based on reducing food intake. An alternative way of treating obesity is greatly needed. From a biological standpoint, obesity results from excess body fat accumulation, which is primarily caused by the oversupply of energy, usually generated by the overconsumption of food. Reducing the oversupply of energy by reducing the efficiency of the energy generation process would be a novel approach to obesity management. The results described above suggest that bromoquinone has great potential to reduce obesity. Experimentation on the effects of bromoquinone on live animals and humans should be conducted to determine if this approach is, in fact, viable.

Although bromoquinone inhibits cell growth in all the cell lines tested, its effectiveness does vary among different cell lines. Without being bound by any particular theory, this could be due to the different concentrations of native coenzyme Q in the different cell lines, as it is possible that a greater concentration of native coenzyme Q mutes the effect of bromoquinone. If that is the case, the effect of bromoquinone on cell growth might be enhanced if it is used together with cholesterol-lowering drugs (such as Crestor or Lipitor). This is because they are also known to decrease the biosynthesis of native coenzyme Q. It is also conceivable that fast-growing cells, such as cancer cells, may be more affected by bromoquinone than the slower-growing ones. If that is the case, bromoquinone may also have potential application in cancer treatment.

### Animal Studies

The effects of 6-Br-Q₀C₁₀ (Formula 1) on the growth of rats was investigated. 6-Br-Q₀C₁₀ was dissolved in different carriers, such as CMC, ethanol, and mixture of oil and butter, and then fed to rats. At lower concentrations, 6-Br-Q₀C₁₀ showed no toxicity and but little effect on growth as measured body weight gain over a period of time. When higher concentrations of 6-Br-Q₀C₁₀ (0.5 mg/IG) were given to rats in 0.3 mL of oil/butter (67%/33%) mixture for a period, a significant 30-40% reduction in body weight gain was observed as compared to the rats that only received the carrier mixture.

### Experimental

### Materials

6-Br-Q₀C₁₀: Crude 6-Br-Q₀C₁₀ was available in the laboratory. It was further purified to homogeneity using thin layer chromatography. The purity of 6-Br-Q₀C₁₀ was confirmed by its absorption peak at 303 nm and the concentration was determined spectrophotometrically, using a millimolar extinction coefficient of 20 cm⁻¹ (13), Carboyxmethyl cellulose was obtained from Amazon. Olive oil and unsalted butter were obtained from the local supermarket. 9 Male F344 (born on Sep. 28, 2023) were purchased from Evigo RMS, Inc, 8520 Allison Pointe Blvd, Suite 4000, Indianapolis, Indiana 46250 on Oct. 17, 2023.

### Methods

The rats were divided into three groups, three rats in each group. We followed their growth by body weight gained daily until Nov. 5, 2023, and then one group of rats received the 6-Br-Q₀C₁₀ treatment, and the second group received only the carrier media used to dissolve the 6-Br-Q₀C₁₀, and the third group was used as control without any treatment. All rats had free access to rat chow and water.

Various concentrations of 6-Br-Q₀C₁₀ were dissolved in 1% and 1.5% CMC, 40% ethanol, and a mixture of oil and unsalted butter (67:33). 6-Br-Q₀C₁₀ solutions and carriers were given to the rats by IG in a volume of 0.3 mL daily. The body weights of the rats were measured and recorded individually and as a group. JACUC Protocol No: SC2101

### Results

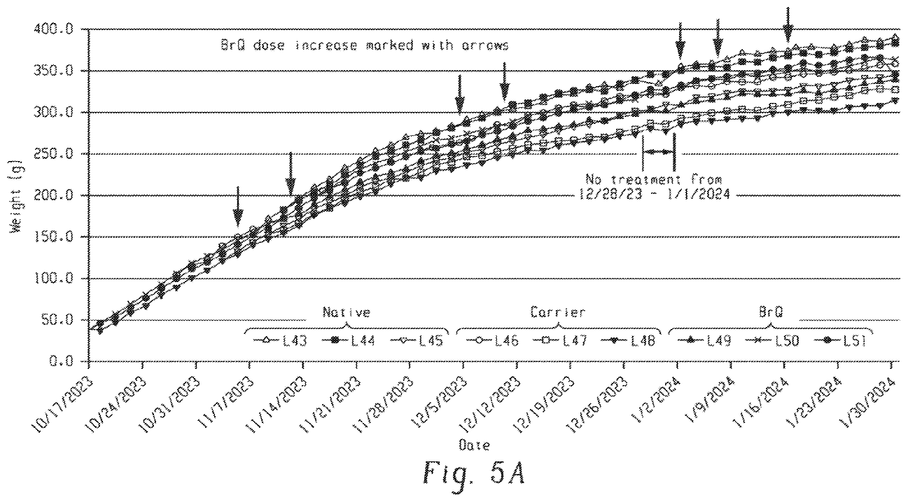

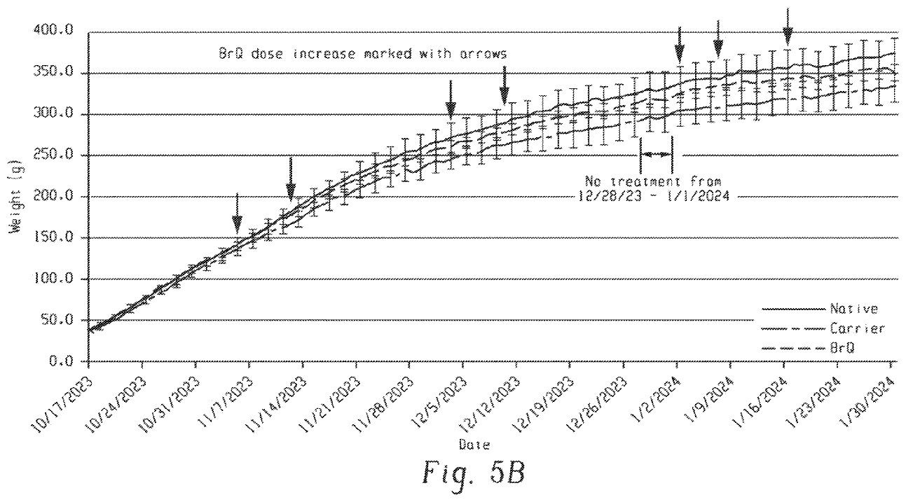

FIG. 5 shows the growth (weight gain) from Nov. 5, 2023 to Feb. 3, 2024 of the three groups of rats, individually (FIG. 5A, Weight gain of all rats) and as group average (FIG. 5B, Group average weight gain of the control, carrier and BrQ treated rats). All of them show typical rat growth characteristics as per the vender's report.

The amounts of 6-Br-Q₀C₁₀ used ranged from 30 ug to 540 ug per rat (Table 3).

TABLE 3
Amounts of 6-Br-Q₀C₁₀ (mg/rat) and carriers used.

| Date | 6-Br-Q₀C₁₀ (mg/rat) | Carrier |
|---|---|---|
| 11/11 | 0.03 | 1% CMC |
| 11/18 | 0.06 | 1% CMC |
| 11/25 | 0.13 | 1.5% CMC |
| 12/3 | 0.23 | 1.5% CMC |
| 1/2 | 0.38 | 40% ethanol |
| 1/16 | 0.54 | Oil/butter (67/33) |
| 1/27 | 0.54 | Oil/butter (67/33) |

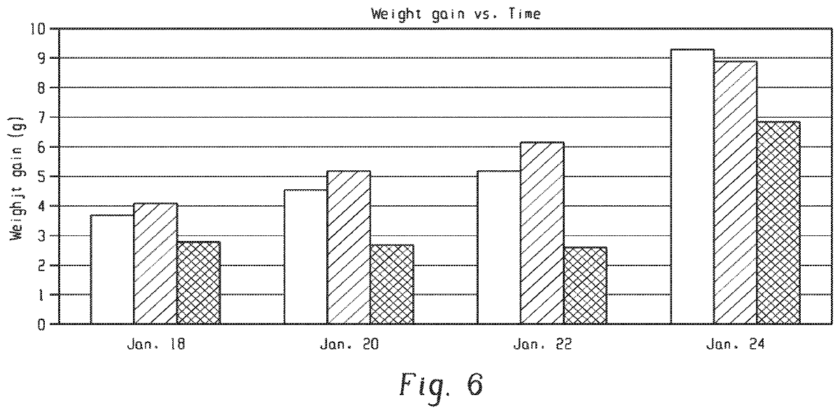

The results indicate that the 6-Br-Q₀C₁₀ treatment has no toxicity effect on rats even at the highest concentrations used. The effect of 6-Br-Q₀C₁₀ on the weight gain of the rats was slight when low concentrations of 6-Br-Q₀C₁₀ were used during the growth period of the rats. However, a slight increase in food and water consumption was observed in the 6-Br-Q₀C₁₀ treated rats. The effect of 6-Br-Q₀C₁₀ on weight gain became more apparent when rats became more mature and higher concentrations of 6-Br-Q₀C₁₀ were used. When 6-Br-Q₀C₁₀ was used together with a high fat diet (the carrier), the effect on weight gain was very striking. A decrease of 30-40% in weight gain was observed. FIG. 6 shows the effect of 6-Br-Q₀C₁₀ on weight gain when the high fat carrier was used.

### Discussion

Animals obtain coenzyme Q₁₀ by two ways, denovo biosynthesis and absorption from food stuffs. It is known that coenzyme Q₁₀ content is higher in growing animals than mature ones (17). When the rats were growing, it is likely that they had higher concentrations of coenzyme Q₁₀ from biosynthesis. Thus, it is understandable that 6-Br-Q₀C₁₀ was less effective in reducing weight gain in the growing rats than the mature ones; also, lower concentrations of 6-Br-Q₀C₁₀ were used when the rats were growing. We anticipate that when higher concentrations of 6-Br-Q₀C₁₀ are used, a more significant decrease in weight gain will be observed.

When the rats were given 0.3 mL of a high fat mixture daily in addition to normal diet, a significant increase in weight gain was observed. This weight gain was decreased by 30-40% when 6-Br-Q₀C₁₀ was used in the mixture, indicating that the 6-Br-Q₀C₁₀ is replacing the normal coenzyme Q₁₀ and participating in the oxidative phosphorylation process in mitochondria of the treated rats. This result agrees with the observation made with 6-Br-Q₀C₁₀ treated isolated beef liver mitochondria. Using NADH linked substrate 6-Br-Q₀C₁₀ treated mitochondria has a P/O ratio of 2 instead of 3. 6-Br-Q₀C₁₀ causes a by-passing of oxidative phosphorylation site 2 because of its high redox-potential.

The effect of 6-Br-Q₀C₁₀ on the reduction of weight gain may be enhanced if it is used together with cholesterol lowering drugs such as Crestor or Lipitor, as they are also known to decrease the biosynthesis of coenzyme Q₁₀. Further experimentation on this aspect is currently planned.

The obesity epidemic is one of the most serious problems that our country faces today (15,16). From a biological standpoint, obesity results from excess body fat accumulation, which is primarily caused by the oversupply of energy, generated from over-consumption of foodstuff. Although a great number of diets, programs and treatments aiming to reduce obesity are available, they are primarily based on the reduction of food intake, which obesity patients may find difficult to accept. An alternative way of treating obesity is greatly needed. Using 6-Br-Q₀C₁₀ or other high midpoint potential Q analogues to reduce energy recovery efficiency offers an innovative alternative to treating obesity.

Also, it follows that fast-growing cells (such as cancer cells) will be more affected by 6-Br-Q₀C₁₀ than the slower growing ones. Cell-line research has already shown that 6-Br-Q₀C₁₀ affects the growth of cancer cell lines. Restricting the energy supply to the cancer cells through treatment with 6-Br-Q₀C₁₀ might slow down their growth. Further research on the effects of 6-Br-Q₀C₁₀ on cancer cells in live animals is contemplated.

Other halogen-Q₀C₁₀, such as 6-Cl-Q₀C₁₀, have also been shown to be effective in reducing cell growth of many commercially available cell lines. It is possible that 6-Cl-Q₀C₁₀ or other halogen formulations may have a similar effect on weight gain and could also be used in obesity management. Experimentation on other formulations is also contemplated.

### REFERENCES

1. Oxidative phosphorylation (1a) “Krebs Cycle.” The Gale Encyclopedia of Science, edited by Katherine H. Nemeh and Jacqueline L. Longe, 6th ed., vol. 4, Gale, 2021, pp. 2512-2514.
2. Trumpower, B. L., and Gennis, R. B. (1994). Energy transduction by cytochrome complexes in mitochondrial and bacterial respiration: The enzymology of coupling electron transfer reactions to transmembrane proton translocation. Annu. Rev. Biochem. 63,675-716,
3. Sazanov, L. A, Hinchliffe P (March 2006). “Structure of the hydrophilic domain of respiratory complex I front Thermus thermophilus”. Science. 311 (5766): 1430-6.
4. Hirst, J. Mitochondrial complex I. Annu. Rev. Biochem. 82, 551-575 (2013).
5. Sun F, Huo X, Zhai Y, Wang A, Xu J, Su D, Bartlam M, Rhao Z. Crystal structure of mitochondrial respiratory membrane protein complex II. Cell. 2005; 121:1043-1057.
6. Gary Cecchini. Function and Structure of complex II of the respiratory chain, Ann, Rev, Biochem, 72, 77-109 (2003) A.
7. Xia, D., Yu, C. A., Kim, H., Xia, J-Z., Kachurin, A. M., Zhang, L. and Yu, L. Deisenho-fer, J. (1997) The Crystal Structure of the Cytochrome bc1 Complex (complex III) from Bovine Heart Mitochondria. Science, 277, 60-66.
8. Iwata S, Lee J W, Okada K, Lee J K, Iwata M, Rasmussen B, Link T A, Ramaswamy S, Jap B K (July 1998). “Complete structure of the 11-subunit bovine mitochondrial cytochrome bc1 complex”. Science. 281 (5373): 64-68.
9. Kyoko Shinzawa-et al. Monomeric structure of an active form of bovine cytochrome c oxidase: PNAS 116 (40) 19945-1995 (2019).
10. Michel H., Behr, J., Kannt, A. Cytochrome c oxidase: structure and spectroscopy. Annu Rev Biophys Biomol Struct 1998; 27:329-356.
11. Senior, AE, Nadanaciva S, Weber J. The molecular mechanism of ATP synthesis by F1F0-ATP synthase. Biochimica et Biophysica Acta (BBA)—Bioenergetics. 2002; 1553 (3): 188-211.
12. Crane F. L. and Barr R. (1985) in Coenzyme Q (Lenaz, G. Edited), John Wiley & Sons.
13. Yu, C. A. and Yu, L. (1982) Syntheses of Biologically Active Ubiquinone Derivatives. Biochemistry 21, 4096-4101.
14. Shunk, C. H., Linn, B. O., Wong, E. L., Wittereich, P. E., Robinson, F. M. and Folkers, K. (1958) J. Am. Chem. Soc, 80, 4753-4756.
15. “Obesity.” Medical Laboratory Observer, vol. 51, no. 4, April 2019, p. 6.
16. Haslam, David W, and W Philip T James. “Obesity.” Lancet (London, England) vol. 366,9492 (2005): 1197-209.
17. Beyer et al. Tissue Coenzyme Qand Protein Concentration Over the Life Span of the Laboratory Rats. Mech. Aging Dev (1985) pp, 267-281.

1. A compound of Formula I, or a salt, solvate, or prodrug thereof: 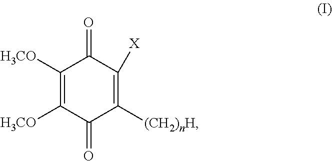
wherein n as an integer from 1 to 15, and X is a halogen.

2. The compound of claim 1, wherein X is selected from Br and Cl.

3. The compound of claim 1, wherein n is 10.

4. The compound of claim 1, having the structure:

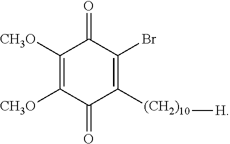

5. A pharmaceutical composition comprising the compound of Formula I, or a salt, solvate, or prodrug thereof, together with a pharmaceutically acceptable carrier.

6. The pharmaceutical composition of claim 4, wherein X is selected from Br and Cl.

7. The pharmaceutical composition of claim 4, wherein n is 10.

8. The pharmaceutical composition of claim 4, wherein the compound of Formula I has the structure:

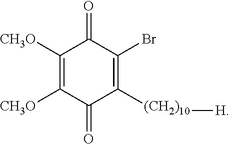

9. A method of treating a disease, disorder, or medical condition in a patient, comprising the step of providing to a patient in need thereof a pharmaceutical composition comprising the compound of Formula I or a salt, solvate, or prodrug thereof, together with a pharmaceutically acceptable carrier.

10. The method of treating a disease, disorder, or medical condition in claim 9, wherein said disease, disorder, or medical condition includes obesity.

11. The method of treating a disease, disorder, or medical condition in claim 9, wherein said disease, disorder, or medical condition includes one or more cancers.

12. The method of treating a disease, disorder, or medical condition of claim 11, wherein the cancer is selected from glioma (glioblastoma), acute myelogenous leukemia, acute myeloid leukemia, myelodysplastic/myeloproliferative neoplasms, sarcoma, chronic myelomonocytic leukemia. non-Hodgkin lymphoma, astrocytoma, melanoma, non-small cell lung cancer, small cell lung cancer, cholangiocarcinomas. chondrosarcoma, colon cancer, colorectal cancer, rectal cancer or pancreatic cancer.

13. The method of claim 9, wherein in the compound of Formula I X is selected from Br and Cl.

14. The method of claim 9 wherein in the compound of Formula I, n is 10.

15. The method of claim 9 wherein the compound of Formula 1 has the structure:

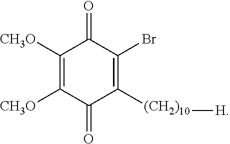

16. The method of claim 9, wherein the patient is a human.
